(19) United States
(12) Patent Application Publication
(10) Pub. No.: US 2010/0048980 A1
(43) Pub. Date: Feb. 25, 2010

(54) ELECTRO-MECHANICAL MASSAGE DEVICE AND WEARABLE MASSAGE APPARATUS

(75) Inventors: Gerrit Jan De Jong, Drachten (NL); Joldert Maria Boersma, Drachten (NL); Pieter Johannes Bax, Drachten (NL); Hermanus Sytse Joseph Altena, Drachten (NL); Erik Van Van Bijssum, Drachten (NL); Andries Symen Kooistra, Wartena (NL); Willem Sjoerd Dijkstra, Witmarsum (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/595,263

(22) PCT Filed: Apr. 14, 2008
(86) PCT No.: PCT/IB2008/051407

§ 371 Date: Oct. 9, 2009

(30) Foreign Application Priority Data
Apr. 18, 2007 (EP) 07106414.1

(51) Int. Cl. A61N 1/00

(52) U.S. Cl. 600/13

(57) ABSTRACT

The invention relates to a massage device (1). The massage device (1) comprises a first support (32), a second support (30) and a deformable connector (36). The first support (32) has a massage face (34). The second support (30) has a protrusion (31) for accommodating an electrical coil (8). During use of the massage device (1), electrical coil 8 conducts an electrical current. First support (32) is provided with an electromagnetic system (15) comprising permanent magnets (14, 24) or one or more electrical coils, magnetically conductible bodies or a combination thereof. First support (32) is connected to second support (30) by a deformable connector (36). As a result of electro-magnetic interaction a force indicated by an arrow (56) is exerted on protrusion (31). The first support (32) may be displaced relative to the second support (30).

### FIELD OF THE INVENTION

The invention relates to an electro-mechanical massage device comprising a first support comprising at least one electromagnetic system and a second support comprising at least one electrical coil for magneto-electrical cooperation with said electromagnetic system, wherein one of the supports is provided with a massage face, wherein, during energizing of said electrical coil, the support provided with the massage face is moveable with respect to the other support.

The invention also relates to a wearable massage apparatus comprising the electro-mechanical massage device.

### BACKGROUND OF THE INVENTION

An electro-mechanical massage device is known from EP 0513508 A2. This EP patent application discloses a massage device provided with a ring-shaped coil and a separate permanent magnet, which can be positioned, together with a part of a body, particularly a finger, into the coil for treatment of that part. The magnet has an axial magnet axis. During treatment the finger is present in a magnetic alternating or pulsating field created in the coil by means of a current source, wherein the magnet moves axially. Another alternative magnet has two axial magnet axes for providing tumbling movements. Yet another magnet has a diametrical magnet axis for creating alternating tilting movements.

For reasons that the magnet and the part of the body must be inside the coil during massage, said coil should be sufficiently large to accommodate the part of the body of a person experiencing the massage. In correspondence to the dimensions of the part of the body to be massaged, the dimensions of the massage device and particularly the coil of the massage device are large.

### SUMMARY OF THE INVENTION

It is an object of the invention to provide an electro-mechanical massage device, which is compact.

According to the invention this object is realized in that the electro-mechanical massage device comprises a deformable connector for connecting the first support to the second support, wherein the first support and the second support are mutually displaceable in a direction substantially parallel to the massage face.

The first support and the second support are connected by the deformable connector. A force is transmittable via the deformable connector between the first support and the second support. Such a force originates from electro-magnetic interaction between an electromagnetic system, e.g. a permanent magnet or a coil or a magnetically conductible body, in the first support and an electrical coil, e.g. an insulated wire which may be wound around or wrapped around a soft-iron body or core, in the second support. Said force is transmitted without the restriction of having the second support, comprising the coil, around and outside the first support, comprising the permanent magnet. The force causes a displacement of the first support relative to the second support in a direction substantially parallel to the massage face. Said displacement may be realized without the restriction of arranging a coil in one support around a permanent magnet in the other support. For this reason a compact electro-mechanical massage device is obtainable.

Additional advantages of having no restriction as described above are that the weight of the massage device may be reduced and that the massage device is wearable.

Yet an additional advantage of the invention is that a natural massage experience is realized during use of the massage device. A natural manual massage may imply a rotating movement of the hand combined with a certain pressure. By providing a mutual displacement of the first support and the second support in a direction substantially parallel to the massage face, this rotating movement is enabled. The device may be applied in a wearable massage apparatus, in which application the electro-mechanical massage device may be applied with a certain pressure to the part of the body experiencing the massage. Thus an effective and satisfying massage is obtainable.

An advantageous embodiment of the device according to the invention is defined in that the at least one electromagnetic system has a permanent magnet and at least one electro-magnetic drive unit, which drive unit includes an electrical coil and the permanent magnet of the at least one electromagnetic system, for imposing a mutual displacement of the electrical coil of the drive unit and the permanent magnet of the drive unit, during energizing of the electrical coil of the drive unit, wherein the mutual displacement of the electrical coil of the drive unit and the permanent magnet of the drive unit contributes to a mutual displacement of the first and the second support in the direction substantially parallel to the massage face.

The magnetic field established by the permanent magnet interacts with the electrical coil. For this reason, the transmission of force between the electrical coil on the one hand and the permanent magnet on the other hand may be very effective. The dimensions of the drive unit comprising the electrical coil and the permanent magnet may be kept small by virtue of the effectiveness of the force transmission in the drive unit.

An advantageous embodiment of the device according to the invention is defined in that at least one electro-magnetic drive unit is provided, which drive unit includes an electrical coil and two permanent magnets, wherein the permanent magnets of the drive unit establish magnetic fields through distinct portions of the electrical coil of the drive unit, for imposing a mutual displacement of the electrical coil of the drive unit and the permanent magnets of the drive unit, during energizing of the electrical coil of the drive unit, wherein the mutual displacement of the electrical coil of the drive unit and the permanent magnets of the drive unit contributes to a mutual displacement of the first and the second support in a direction substantially parallel to the massage face.

The transmission of force between the permanent magnets on the one hand and the electromagnet on the other hand is optimized by establishing two magnetic fields through distinct portions of the electromagnet. Two permanent magnets per electromagnet are less space consuming than three or even more permanent magnets. The magnetic fields, established through distinct portions of the electromagnet, enable efficient generation of electro-mechanical force between the permanent magnets on the one hand and the electromagnet on the other hand. Establishing the direction of the magnetization of the permanent magnets parallel to the coil axis of the electrical coil further contributes to efficient generation of electro-mechanical force between the permanent magnets on the one hand and the electrical coil on the other hand.

An advantageous embodiment of the device according to the invention is defined in that more than one electro-magnetic drive unit is provided, wherein the first support is provided with a pattern of permanent magnets, which permanent magnets are integrated in the drive units, wherein the second support is provided with a configuration of electrical coils, which electrical coils are integrated in the drive units, which configuration of electrical coils is provided for magneto-electrical cooperation with the pattern of permanent magnets.

The pattern of permanent magnets and the corresponding configuration of electromagnets enable a distribution of the force between the first support and the second support over a large massage surface, while keeping the distance between the first support and the second support small relative to the dimensions of the massage surface. Forces between the first support and the second support may be generated in perpendicular directions, which directions are substantially parallel to the massage face. The forces being generatable in perpendicular directions, a vast choice of massage actions is feasible by adjustment of these forces and the resulting displacement between the first support and the second support. The displacement between the first support and the second support has a magnitude that corresponds to the dimensions of a drive unit. By choosing the dimensions of the drive unit corresponding to a desired characteristic magnitude of said displacement, the design of a control unit for controlling the massage action of the electro-mechanical massage device according to the invention is facilitated.

In an advantageous embodiment according to the invention, the drive units are included by electromagnets, comprising the electrical coils of the configuration of electrical coils and magnetically conductive bodies.

The interaction between the permanent magnets and the electrical coils may be amplified by the presence of said magnetically conductive bodies. This contributes to the compactness of the drive units and to the compactness of the electro-mechanical massage device according to the invention.

An advantageous embodiment of the device according to the invention is defined in that the permanent magnets are arranged in a sequence of alternating magnetic polarity.

Permanent magnets are widely available and may be relatively easy to produce. Arranging the permanent magnets in a sequence of alternating magnetic polarity further contributes the compactness of the drive units and to the compactness of the electro-mechanical massage device according to the invention. Materials for suitable permanent magnets are e.g. neodymium iron boron (NdFeB), samarium cobalt (SmCo), Ceramic and Alnico. NdFeB magnets exhibit advantageous properties to obtain a very effective force transmission between the first support and the second support. Ceramic magnets are also known as ferrite magnets (general composition BaFe2O3 or SrFe2O3). They have been commercialized since the 1950s and may be advantageously used due to their low cost. A special form of a ceramic magnet is made by bonding ceramic powder in a flexible binder. Flexible magnetic material is advantageous to provide flexibility to the massage device. The dimension along the magnetic axis or the thickness of such permanent magnets may be kept small, which is beneficial for the compactness of the massage device. The alternating polarity further optimizes force transition between magnets and coil. For reason of the optimized force transition the dimension of the drive unit may be further reduced.

An advantageous embodiment of the device according to the invention is defined in that the first support and the second support are mutually displaceable in two directions, which directions are substantially parallel to the massage face, by arranging the permanent magnets in a checkerboard-like pattern of alternating magnetic polarity.

The repetition in the arrangement of the permanent magnets is beneficial to ease of manufacturing. Furthermore, the checkerboard-like pattern provides two kinds of tracks of permanent magnets of alternating polarity, viz. a first kind and a second kind of tracks. The first kind enables a first displacement of the first support relative to the second support in a first direction, while the second kind of tracks enables a second displacement of the first support relative to the second support in a second direction, which second direction is perpendicular to the first direction. In a number of applications, the mutual displacement of the first support and the second support may be controllable by a control unit or adjustable by an adjustment unit. Ease of controlling or adjusting the mutual displacement may be served by the presence of said first direction and said second direction, which directions are orthogonal and independently addressable by such a control unit or an adjustment unit. By properly driving the drive units by the control unit even a rotation of the first support relative to the second support around an axis perpendicular to the massage face is possible.

An advantageous embodiment of the device according to the invention is defined in that the first support and the second support are mutually displaceable in two directions, which directions are substantially parallel to the massage face, and wherein the first support and the second support are mutually rotatable around an axis of rotation, which axis of rotation is substantially perpendicular to the massage face, by arranging the permanent magnets in a ring-shaped pattern of alternating magnetic polarity. The ring-shaped pattern comprises at least one ring of magnets of alternating magnetic polarity.

On top of the advantages of the checkerboard-like pattern, the ring-shaped pattern has extended ease of control, because the rotation around the axis of rotation is a degree of freedom in the mutual displacement of the first support and the second support, which degree of freedom is implicitly present in the ring-shaped pattern.

An advantageous embodiment of the device according to the invention is defined in that the permanent magnets are arranged in concentric rings of the ring-shaped pattern.

By arranging the permanent magnets in concentric rings, complexity of a control unit is further avoided in imposing a rotation of the first support relative to the second support, because the angle of rotation between each ring of electrical coils and each corresponding ring of permanent magnets is the same for all concentric rings.

The wearable massage apparatus according to the invention is characterized in that it comprises the electro-mechanical massage device according to the invention.

### BRIEF DESCRIPTION OF THE DRAWINGS

These and other aspects of the appliance according to the invention will be exemplarily elucidated and described with reference to the drawings, in which:

FIG. 1 is a sectional view of a part of an embodiment of the massage device according to the invention.

FIG. 2 is a perspective view showing a detail of the embodiment of FIG. 1.

FIG. 3 is a sectional view of a part of an embodiment according to the invention.

FIGS. 4a and 4b are top views of a part of an embodiment of the massage device according to the invention.

FIGS. 5a, 5b and 5c are top views of a part of an embodiment of the massage device according to the invention.

FIGS. 6a, 6b and 6c are top views of a part of an embodiment of the massage device according to the invention.

### DETAILED DESCRIPTION OF PREFERRED EMBODIMENTS

It is to be noted that all disclosed embodiments are schematically depicted.

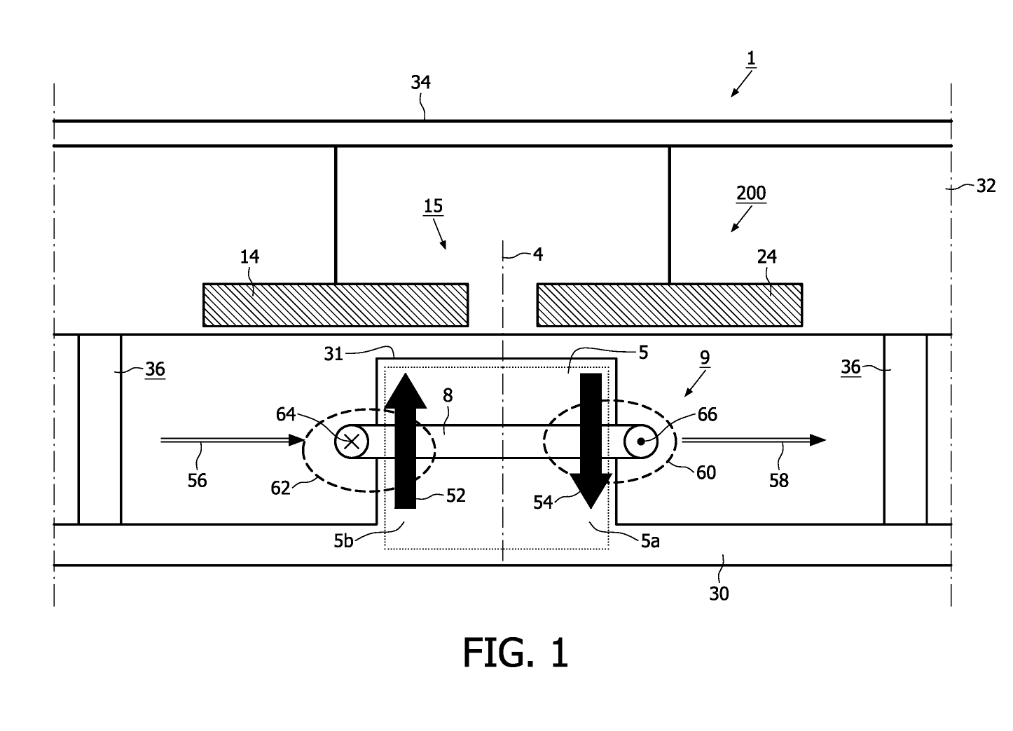

In FIG. 1 a sectional view of a part of an embodiment of the massage device according to the invention is depicted. The embodiment according to FIG. 1 comprises a part of an electro-mechanical massage device 1. The massage device 1 comprises a first support 32, a second support 30 and a deformable connector 36. The first support 32 has a massage face 34. The massage face 34 could have been provided to the second support 30 as well, without limiting the functionality of the electro-mechanical massage device 1.

Second support 30 has a protrusion 31 for accommodating an electrical coil 8 of an electromagnet 9. The protrusion 31 comprises a magnetically conductible body 5 (indicated by the dotted lines). Body 5 of electromagnet 9 may comprise commonly known magnetically conductible materials such as e.g. soft-iron, cobalt, nickel and certain alloys of these metals. Electrical coil 8 has a coil axis 4. During use of the massage device, electrical coil 8 conducts an electrical current. The direction of the electrical current in portions 60 and 62 of the electrical coil 8 is according to the direction as indicated by arrow tail 64 and arrow head 66, respectively.

First support 32 is provided with an electromagnetic system 15, comprising permanent magnets 14 and 24. The electromagnetic system 15 may also comprise one or more electrical coils, magnetically conductible bodies a combination thereof. First support 32 is connected to second support 30 by a deformable connector 36. Permanent magnets 14 and 24 are two-poled permanent magnets, establishing magnetic fields through the electromagnet 9, comprising the electrical coil 8 and the magnetically conductible body 5 of protrusion 31. Permanent magnet 14 establishes a first magnetic field as indicated by arrow 52 through portion 62 of electromagnet 9 and a first part 5a of the magnetically conductible body 5, while permanent magnet 24 establishes a second magnetic field as indicated by arrow 54 through portion 60 of electromagnet 9 and a second part 5b of the magnetically conductible body 5. The magnetic fields of magnets 14 and 24 are of opposite direction as indicated by arrows 52 and 54.

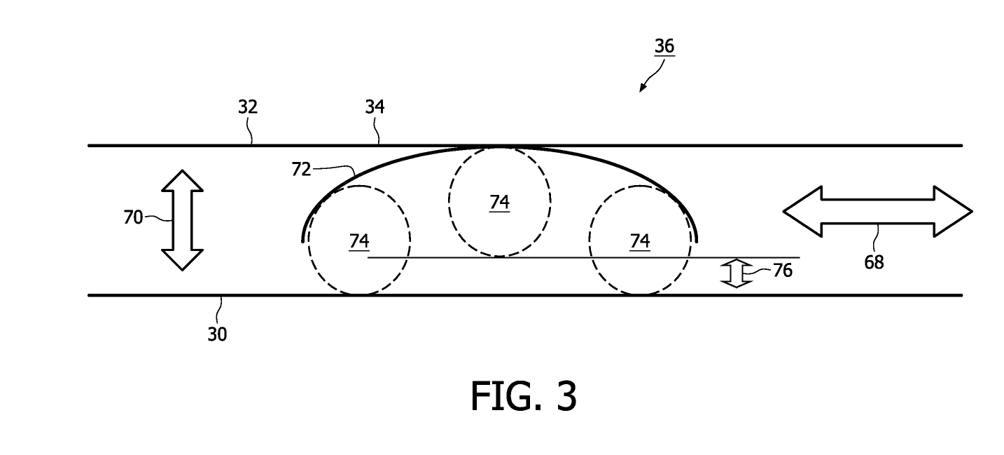

The first magnetic field through portion 62, indicated by arrow 52, and the electrical current through portion 62 cause a first electromagnetic interaction between magnet 14 and portion 62 of electrical coil 8 and body 5, i.e. between magnet 14 and electromagnet 9. As a result of this first interaction a first electro-magnetic force is exerted on protrusion 31 as indicated by arrow 56. The second magnetic field through portion 60, indicated by arrow 54, and the electrical current through portion 60 cause a second electro-magnetic interaction between magnet 24 and portion 60 of electrical coil 8 and body 5, i.e. between magnet 24 and electromagnet 9. As a result of this second interaction a second electro-magnetic force is exerted on protrusion 31 as indicated by arrow 58. The first electro-magnetic force and the second electro-magnetic force have the same direction. This is indicated by arrows 56 and 58 pointing in the same direction. The first electro-magnetic force will cause a first reaction force (not indicated in FIG. 1) on permanent magnet 14 and the second electro-magnetic force will cause a second reaction force (not indicated in FIG. 1) on magnet 24. As a result of these reaction forces, the first support 32 may be displaced relative to the second support 30, because the connector 36, further also called interconnection 36, between the first support 32 and the second support 30 is deformable. The connector 36 may have the form of e.g. a connector body between the first support 32 and the second support 30, which body is elongated in a direction perpendicular to the massage face 34 and may be made of a material that is resilient to allow for the displacement between the first support 32 and the second support 30, e.g. a leaf spring made of a polymer or spring metal. An advantageous embodiment of connector 36 is depicted in FIG. 3. If the interconnection 36 is deformable in a direction perpendicular to the coil axis 4, a mutual displacement between the first support and the second support will be parallel or mainly parallel to the massage face 34. The generation of the electro-magnetic forces as described here above is not limited to magnetic fields of opposite direction through distinct portions of the coil. Such forces may also be generated, e.g. by providing different magnetic fields having the same directions through the portions 60 and 62 of the coil or e.g. by providing asymmetry in the portions of the coil being fluxed by the magnetic field or fields. Also, it is possible to have only one magnetic field fluxing a portion of an electrical coil. For example, permanent magnet 14 may be replaced by a piece of magnetically conductible material to ascertain sufficient magnetic flux of permanent magnet 24 through protrusion 31. In a basic and simple embodiment according to the invention it may be sufficient to have a magnetically conductible body over-wound by an electrical coil facing a permanent magnet. For this reason, the schematic depiction of an embodiment of the invention given in FIG. 1 is not limiting the scope of the invention.

By inverting the electrical current through the electrical coil 8, the electro-magnetic forces and the mutual displacement between the first support 32 and the second support 30 will be inverted. Application of an alternating electrical current through electrical coil 8 will provoke a reciprocating motion of the first support 32 relative to the second support 30.

It is also possible to keep the current through the coil 8 constant to apply a constant massage force to a portion of the body experiencing the massage. In the situation of applying such a constant force the displacement between the first support and the second support will stay fixed, e.g. to provide a constant support or correction to the portion of the body that is actuated by the electro-mechanical massage device according to the invention.

FIG. 1 shows a possible arrangement of an electromagnetic system 15 in the first support 32. Said electromagnetic system 15 may comprise any set of elements chosen from the group comprising an electrical coil, a magnetically conductible body and a permanent magnet. Thus it is possible, that the permanent magnets 14 and 24 are replaced by two electrical coils, wound around a soft-iron body as a magnetically conductible body or by a magnetically conductible body and an electrical coil and a permanent magnet. At least one electrical coil is present in the second support 30 to enable energizing of the massage device 1.

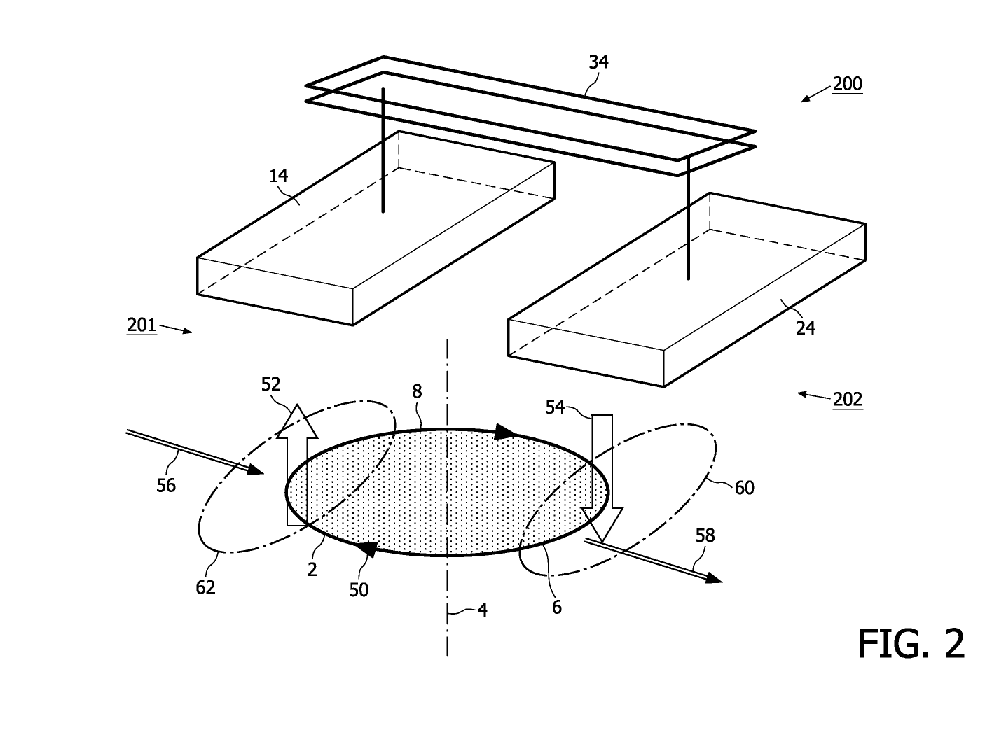

Electromagnet 9, comprising the electrical coil 8 and magnetically conductible body 5, and permanent magnets 14 and 24 may be provided to electro-magnetic drive units 200 as schematically depicted in FIG. 2 in perspective view. The first support, the second support and the magnetically conductible body 5 are not visible in FIG. 2. The permanent magnet 14 is during use in interaction with portion 62 of electromagnet 9 (not shown) and its magnetically conductible material inside coil 8 (indicated by the dotted surface), whereby a force, indicated by arrow 56, is generated. The combination of permanent magnet 14 and electrical coil 8 may be regarded as a first drive unit 201, which first drive unit causes a mutual displacement between the first support and the second support resulting in a motion of the massage face 34 relative to the electrical coil 8. The permanent magnet 24 is in interaction with portion 60 of coil 8. A force, indicated by arrow 58, is generated. The combination of permanent magnet 14 and electrical coil 8 may be regarded as a second drive unit 201, which first drive unit causes a mutual displacement between the first support and the second support contributing to the motion or displacement of the massage face 34 relative to the electrical coil 8. In a basic arrangement, a drive unit comprises a combination of a portion of an electrical coil around a magnetically conductible material and a permanent magnet. In a more elaborate arrangement such a drive unit may also comprise a combination of an electrical coil and two permanent magnets as depicted in FIG. 2. It may however also be possible that the drive unit comprises one permanent magnet and two electrical coils, wherein the magnetic field of the magnet effluxes portions of the two electrical coils. To optimize the interaction between the first support and the second support, it is advantageous to have sufficient coverage of the area of the first support by permanent magnets.

In FIG. 3 an interconnection 36 between a first support 32 and a second support 30 is schematically depicted. An electro-magnetic drive unit (not shown) causes a first reciprocating motion of the first support relative to the second support. The first reciprocating motion is indicated by an arrow 68 and parallel to a massage face 34. The deformable connector 36 comprises a bowl or bowl-like body 72 and a ball bearing 74. During the reciprocating motion the ball bearing causes a reciprocating motion of the first support relative to the second support in a direction perpendicular to the massage face 34. The curvature of the bowl 72 determines the distance between the first support 32 and the second support 30 as a function of the mutual displacement between said supports parallel to the massage face 34. By tuning the curvature of the bowl 72 and the size of the ball bearing 72, the massage motion of the massage face relative to the second support 30 is tuneable in a direction perpendicular to the massage face 34. The resulting motion of the massage 34 relative to the second support 30 enables a massage having a component parallel to the massage face 34 and also having a component perpendicular to the massage face. The amplitude of the perpendicular component is indicated by an arrow 76. An advantage of the embodiment of a flexible connector as schematically depicted in FIG. 3 is, that the deformation of the connector is realized by a displacement of the ball bearing 74 relative to bowl 72. For this reason no material deformation is needed, which is beneficial for the lifetime of the device.

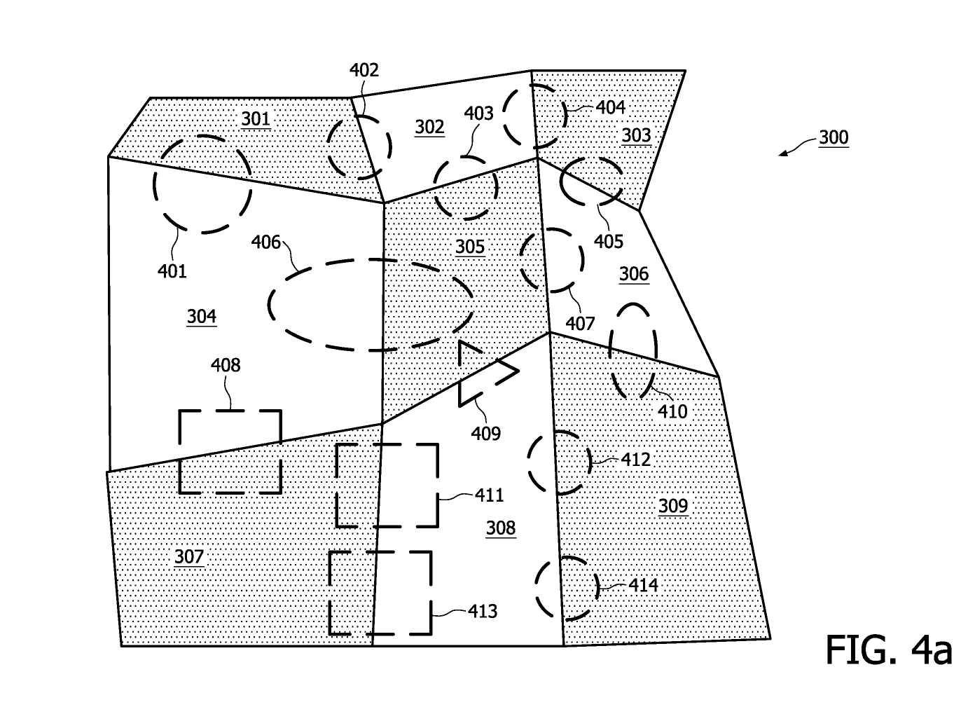

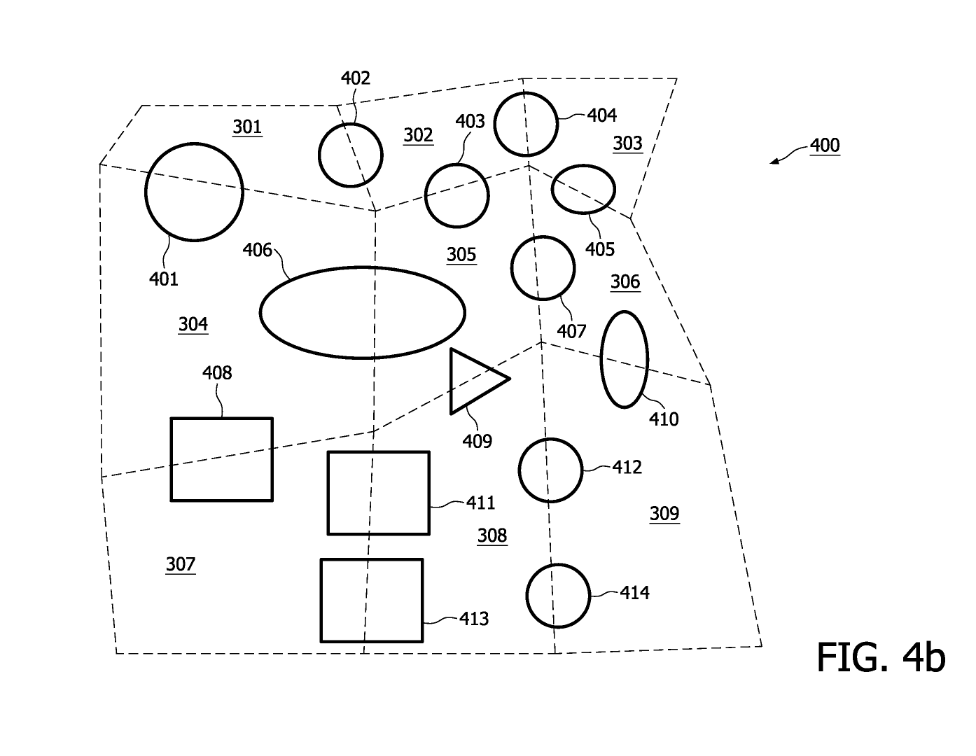

FIG. 4a schematically depicts a pattern 300 of permanent magnets 301-309. The magnets may be two-poled permanent magnets. Permanent magnets 301 to 309 are provided to a first support for electro-magnetic cooperation with electric coils 401 to 414. The electric coils 401 to 414 are not provided to the first support and are indicated by dashed lines. As indicated here above, the electrical coils of the electromagnets are wound around magnetically conductible bodies to obtain interaction between the pattern of permanent magnets 300 and the configuration of electrical coils. Various drive units may be discerned, such as the combination of permanent magnets 305 and 306 with round electrical coil 407. Another drive unit is established by triangular electrical coil 409 and permanent magnets 305 and 308. Rectangular coil 411 provides a drive unit in combination with magnets 307 and 308. The same magnets 307 and 308 provide yet another drive unit in combination with rectangular coil 413. In FIG. 4b the configuration of electrical coils 400, corresponding to the pattern of permanent magnets of FIG. 4a is indicated. In FIGS. 4a and 4b it is emphasized, that the patterns of the magnets 300 and the corresponding configuration of electrical coils may have innumerable manifestations, all falling within the scope of the invention.

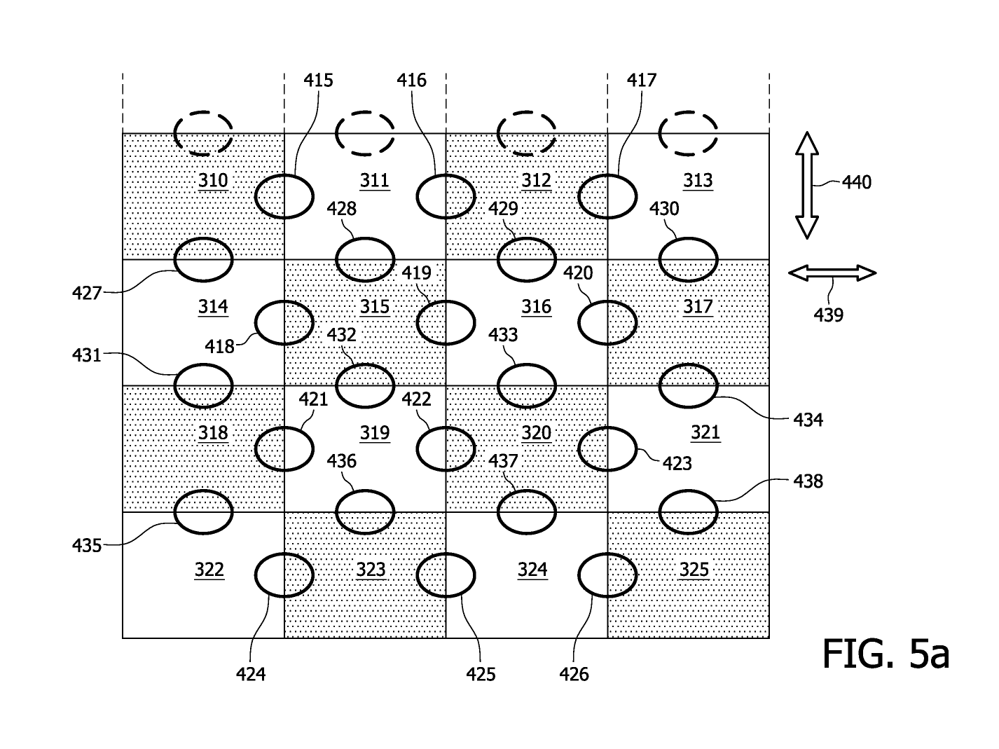

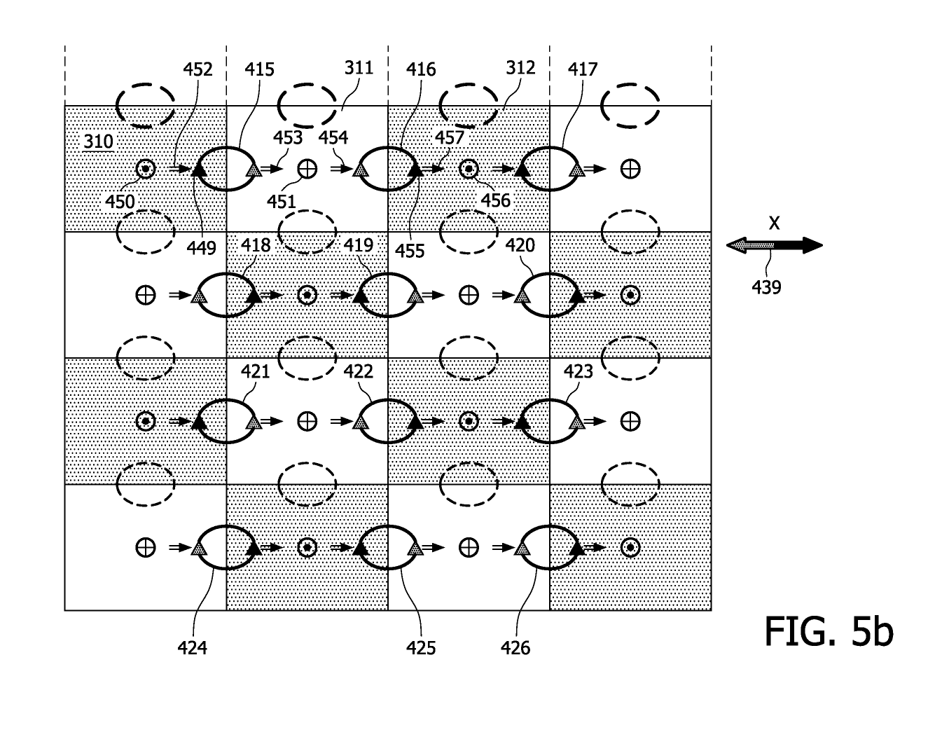

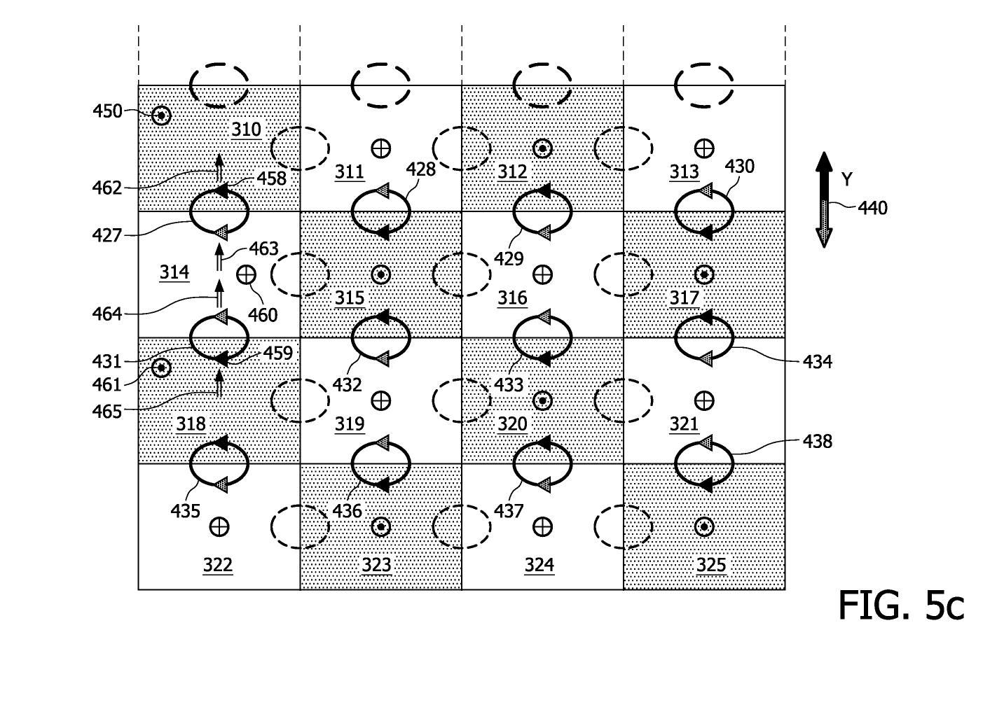

FIG. 5a shows yet another embodiment by arranging two-poled permanent magnets 310-325 in a checkerboard-like pattern of alternating magnetic polarity. Permanent magnets 310, 312, 315, 317, 318, 320, 323 and 325 provide magnetic fields for electro-magnetic interaction with electrical coils 415 to 438. The directions of these magnetic fields are indicated in FIGS. 5b and 5c by the arrow heads, e.g. arrow head 450 and 456. Permanent magnets 311, 313, 314, 316, 319, 321, 322 and 324 also provide magnetic fields for electro-magnetic interaction with electrical coils 415 to 438, however, of opposite direction as indicated by the arrow tails 451 in FIGS. 5b and 460 in FIG. 5c. For reason of clarity not all arrow heads and arrow tails have reference signs.

A configuration of coils 415 to 438 (FIG. 5a), corresponding to the pattern of the permanent magnets may be controlled as indicated in FIG. 5b to generate a reciprocating motion between the pattern of permanent magnets, comprised by a first support (not shown) and the configuration of coils comprised by a second support (not shown), the reciprocating motion being according to the direction as indicated by double headed arrow 439. In this example of controlling the current in the coils 415 to 438, only coils 415 to 426 are carrying an electrical current. In the example as depicted in FIG. 5b, electrical coils 427 to 438 are not operated. The direction of the electrical currents in coils 415 to 426 is indicated by triangles, e.g. triangle 449 and 455.

Permanent magnets 310 and 311 and electrical coil 415 establish a drive unit. The electrical current in coil 415 in the direction as indicated by triangle 449 interacts with the magnetic fields as indicated by arrow head 450 and arrow tail 451 of permanent magnets 310 and 311 respectively. As a result forces are exerted on coil 415 as indicated by arrows 452 and 453. Similar drive units, generating corresponding forces, are formed by coil 417 and magnets 312 and 313, coil 419 and magnets 315 and 316, coil 421 and magnets 318 and 319, etc.

Permanent magnets 311 and 312 and electrical coil 416 also establish a drive unit. The electrical current in coil 416 in the direction as indicated by triangle 455 interacts with the magnetic fields as indicated by arrow tail 451 and arrow head 456 of permanent magnets 311 and 312 respectively. As a result forces are exerted on coil 416 as indicated by arrows 454 and 457. Similar drive units, generating corresponding forces, are formed by coil 418 and magnets 314 and 315, coil 420 and magnets 316 and 317, coil 422 and magnets 319 and 320, etc. As can be seen from FIG. 5b, the direction of the current in the electrical coils 415, 417, 419, 421, 423 and 425 is clockwise while the direction of the current in coils 416, 418, 420, 422, 424 and 426 is counter clockwise. This knowledge may be conveniently used to wind the coils accordingly, to simplify an electric control unit (not shown), which control unit provides the electric currents to said coils.

The configuration of coils 415 to 438, corresponding to the pattern of the permanent magnets may also be controlled as indicated in FIG. 5c to generate a reciprocating motion between the pattern of permanent magnets, comprised by a first support (not shown) and the configuration of coils comprised by a second support (not shown), the reciprocating motion being according to the direction as indicated by double headed arrow 440. The resulting motion of FIG. 5c is perpendicular to the motion of FIG. 5b. In this example of controlling the current in the coils 415 to 438, only coils 427 to 438 are carrying an electrical current. In the example as depicted in FIG. 5c, electrical coils 415 to 426 are not operated. The direction of the electrical currents in coils 427 to 438 is indicated by triangles, e.g. triangle 458 and 459.

Permanent magnets 310 and 314 and electrical coil 427 establish a drive unit. The electrical current in coil 427 in the direction as indicated by triangle 458 interacts with the magnetic fields as indicated by arrow head 450 and arrow tail 460 of permanent magnets 310 and 314 respectively. As a result forces are exerted on coil 427 as indicated by arrows 462 and 463. Similar drive units, generating corresponding forces, are formed by coil 429 and magnets 312 and 316, coil 432 and magnets 315 and 319, coil 434 and magnets 317 and 321, etc.

Permanent magnets 314 and 318 and electrical coil 431 also establish a drive unit. The electrical current in coil 431 in the direction as indicated by triangle 459 interacts with the magnetic fields as indicated by arrow tail 460 and arrow head 461 of permanent magnets 314 and 318 respectively. As a result forces are exerted on coil 431 as indicated by arrows 464 and 465. Similar drive units, generating corresponding forces, are formed by coil 428 and magnets 311 and 315, coil 433 and magnets 316 and 320, coil 430 and magnets 313 and 317, etc. As can be derived from FIG. 5c, the direction of the current in the electrical coils 427, 429, 431, 433, 435 and 437 is counter clockwise while the direction of the current in coils 428, 430, 432, 434, 436 and 438 is clockwise. This knowledge may be conveniently used to wind the coils accordingly, to simplify the electric control unit (not shown).

In the checkerboard-like arrangement of FIGS. 5a, 5b and 5c all coils may be operated simultaneously to generate any combination of motions in the perpendicular directions as indicated by double headed arrows 439 and 440.

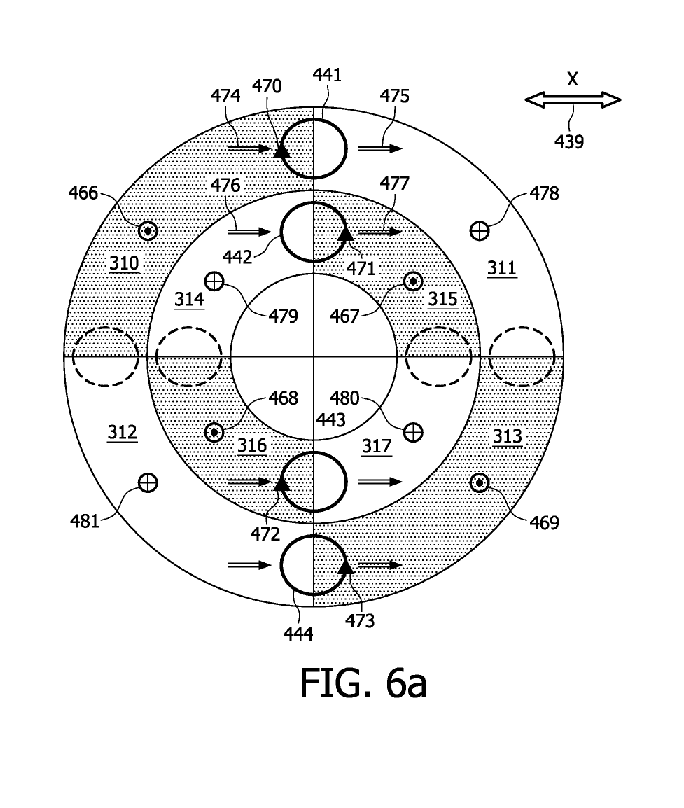

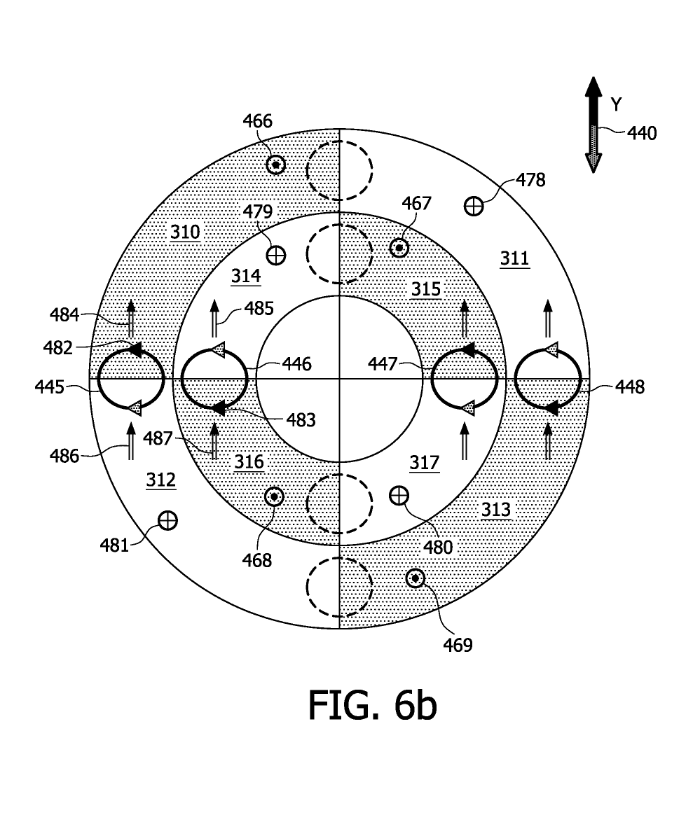

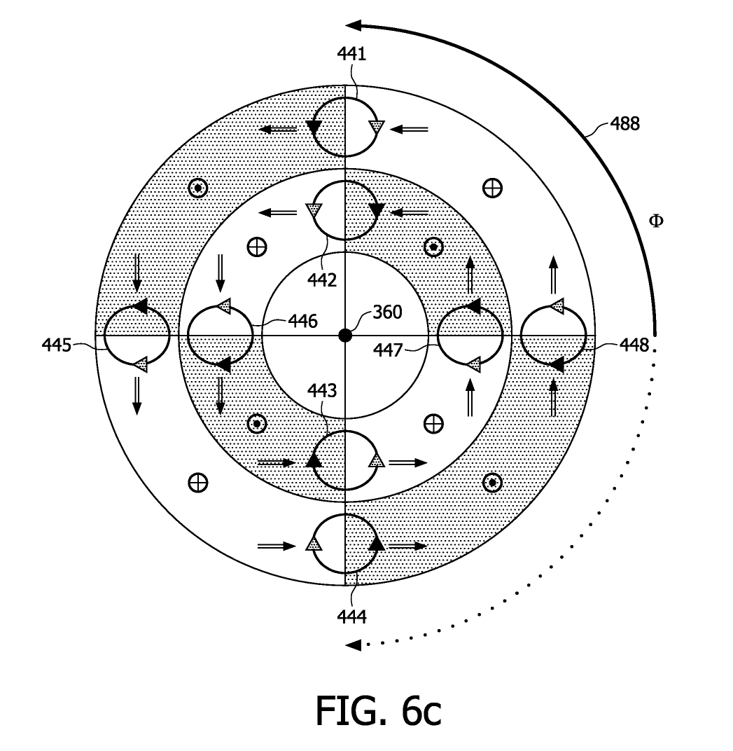

FIGS. 6a, 6b and 6c show another embodiment by arranging the two-poled permanent magnets in a ring-like pattern of alternating magnetic polarity. Permanent magnets 310, 315, 316 and 313 provide magnetic fields at the level of electrical coils 441 to 444. The directions of these magnetic fields are indicated in FIGS. 6a, 6b and 6c by the arrow heads 466 to 469. Permanent magnets 311, 314, 317 and 312 also provide magnetic fields at the level of electrical coils 441 to 444, however, of opposite direction as indicated by the arrow tails 478 to 481.

The configuration of coils 441 to 444 may be operated to conduct a current as indicated by the triangles 470 to 473 (FIG. 6a). If an alternating current is applied to coils 441 to 444, a reciprocating motion between the pattern of permanent magnets, comprised by a first support (not shown) and the configuration of coils comprised by a second support (not shown) will result, according to the electro-magnetic interaction described here above. The reciprocating motion will in X-direction as indicated by double headed arrow 439.

The configuration of coils 445 to 448 may be operated to conduct a current as indicated by the triangles in coils 445 to 448 (FIG. 6b). If once again an alternating current is applied to coils 445 to 448, a reciprocating motion between the pattern of permanent magnets, comprised by a first support (not shown) and the configuration of coils comprised by a second support (not shown) will result, according to the electro-magnetic interaction described here above. The reciprocating motion will in Y-direction as indicated by double headed arrow 440.

In FIG. 6c a situation is depicted, wherein all coils are operated. The direction of the electrical currents in the configuration of coils is addressed to generate forces, having a rotating action, around centre of rotation 360. By the arrangement as depicted in FIG. 6c a rotational degree of freedom is provided as indicated by the curved arrow 448.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. Devices, elements and components, known per se, have not been described in detail, as the skilled person is familiar with the matter. In the claims, the word “comprising” does not exclude other elements or steps, and the indefinite article “a” or “an” does not exclude a plurality. A single mechanism or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. An electro-mechanical massage device comprising:
a first support comprising at least one electromagnetic system and
a second support comprising at least one electrical coil for magneto-electrical cooperation with said electromagnetic system, wherein one of the supports is provided with
a massage face, wherein, during energizing of said electrical coil, the support provided with the massage face is moveable with respect to the other support, characterized by
a deformable connector for connecting the first support to the second support, wherein the first support and the second support are mutually displaceable in a direction substantially parallel to the massage face.

2. An electro-mechanical massage device according to claim 1, wherein the at least one electromagnetic system has a permanent magnet and wherein at least one electro-magnetic drive unit is provided, which drive unit includes an electrical coil and the permanent magnet of the at least one electromagnetic system, for imposing a mutual displacement of the electrical coil of the drive unit and the permanent magnet of the drive unit, during energizing of the electrical coil of the drive unit, wherein the mutual displacement of the electrical coil of the drive unit and the permanent magnet of the drive unit contributes to a mutual displacement of the first and the second support in the direction substantially parallel to the massage face.

3. An electro-mechanical massage device according to claim 1, wherein at least one electro-magnetic drive unit is provided, which drive unit includes an electrical coil and two permanent magnets, wherein the permanent magnets of the drive unit establish magnetic fields through distinct portions of the electrical coil of the drive unit, for imposing a mutual displacement of the electrical coil of the drive unit and the permanent magnets of the drive unit, during energizing of the electrical coil of the drive unit, wherein the mutual displacement of the electrical coil of the drive unit and the permanent magnets of the drive unit contributes to a mutual displacement of the first and the second support in a direction substantially parallel to the massage face.

4. An electro-mechanical massage device according to claim 2, having more than one electro-magnetic drive unit, wherein the first support is provided with a pattern of permanent magnets, which permanent magnets are integrated in the drive units, wherein the second support is provided with a configuration of electrical coils, which electrical coils are integrated in the drive units, which configuration of electrical coils is provided for magneto-electrical cooperation with the pattern of permanent magnets.

5. An electro-mechanical massage device according to claim 4, wherein the drive units are included by electromagnets, comprising the electrical coils of the configuration of electrical coils and magnetically conductive bodies.

6. A device according to claim 4, wherein the permanent magnets of the pattern of permanent magnets are arranged in a sequence of alternating magnetic polarity.

7. An electro-mechanical massage device according to claim 6, wherein the first support and the second support are mutually displaceable in two directions, which directions are substantially parallel to the massage face, by arranging the permanent magnets in a checkerboard-like pattern of alternating magnetic polarity.

8. An electro-mechanical massage device according to claim 6, wherein the first support and the second support are mutually displaceable in two directions, which directions are substantially parallel to the massage face, and wherein the first support and the second support are mutually rotatable around an axis of rotation, which axis of rotation is substantially perpendicular to the massage face, by arranging the permanent magnets in a ring-shaped pattern of alternating magnetic polarity.

9. An electro-mechanical massage device according to claim 8, wherein the permanent magnets are arranged in concentric rings of the ring-shaped pattern.

10. A wearable massage apparatus comprising the electro-mechanical massage device according to claim 1.
